SEQUENCE LISTING (12 sequences)

SEQ ID NO: 1 - protein, 269 aa, synthetic construct
DVSQDLFDQFNLFAQYSAAAYCAKNNDAPAGAIVTCRGSICPEVEKADATFLYSFEDSGVGDVTGFLALDNTNRLIVLSFRGSRSLENWIGNINMDLKGIDDICSGCKGHDGFTSSWRSVANTLTQQVQNAVREHPDYRVVFTGHSLGGALATVAGASLRGNGYDIDVFSYGAPRVGNRAFAEFLTAQTGGTLYRITHTNDIVPRLPPRELGYSHSSPEYWITSGTLVPVRRRDIVKVEGIDSTDGNNQPNTPDIAAHFWYFGSIGTCL

SEQ ID NO: 2 - protein, 269 aa, synthetic construct
DVSQDLFDQFNLFAQYSAAAYCAKNNRAPAGAIVTCRASICPEVEKADATFLYSFEDSGVGDVTGFLALDNTNRLIVLSFRGSRSLENWIGNINMELKGIDDICSGCKGHAGFTSSWRSVANTLTQQVQNAVREHPDYRVVFTGHSLGGALATVAGASLRGNKYDIDVFSYGAPRVGNRAFAEFLTAQTGGTLYRITHTNDIVPRLPPRELGYSHSSPEYWITSGTLVPVRRRDIVKVEGIDSTDGNNQPNTPSITAHFWYFGSIGTCL

SEQ ID NO: 3 - DNA, 1077 bp, synthetic construct
  source 1..1077: mol_type = other DNA
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctcttgagaaaagagaggctgaagctgatgtttctcaagatttgtttgatcaatttaatttgtttgctcaatattctgctgcagcttattgtgctaaaaataatagggctcctgctggtgctattgttacttgtagagcttctatttgtcctgaagttgaaaaagctgatgctacttttttgtattcttttgaagattctggtgttggtgatgttactggttttttggctttggataatactaatagattgattgttttgtcttttagaggttctaggtcattagaaaattggattggtaatattaacatggaattaaaaggtattgatgatatttgttctggttgtaaaggtcatgctggttttacttcttcttggaggtctgttgctaatactttgactcaacaagttcaaaatgctgttagagaacatcctgattatagagttgtttttactggtcattctttgggtggtgctttggctactgttgctggtgcttctttgagaggtaataaatatgatattgatgttttttcttatggtgctccaagagttggtaatagagcttttgctgaatttttgactgctcaaactggtggtactttgtatagaattactcatactaatgatattgttccaagattgccaccaagagaattgggttattctcattcttctcctgaatattggattacttctggtactttggttcctgttagaaggagagatattgttaaagttgaaggtattgattctactgatggtaataatcaaccaaatactccatctattactgctcatttttggtattttggttctattggtacttgtttgtaa

SEQ ID NO: 4 - protein, 269 aa, synthetic construct
DVSQDLFDQFNLFAQYSAAAYCAKNNRAPAGAIVTCRASICPEVEKADATFLYSFEDSGVGDVTGFLALDNTNRLIVLSFRGSRSIENWIGNLNFELKGIDDICSGCKGHAGFTSSWRSVANTLTQQVQNAVREHPDYRVVFTGHSLGGALATVAGASLRGNKYDIDVFSYGAPRVGNRAFAEFLTAQTGGTLYRITHTNDIVPRLPPRELGYSHSSPEYWITSGTLVPVRRRDIVKVEGIDSTDGNNQPNTPSITAHFWYFGSIGTCL

SEQ ID NO: 5 - DNA, 1077 bp, synthetic construct
  source 1..1077: mol_type = other DNA
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctcttgagaaaagagaggctgaagctgatgtttctcaagatttgtttgatcaatttaatttgtttgctcaatattctgctgcagcttattgtgctaaaaataatagggctcctgctggtgctattgttacttgtagagcttctatttgtcctgaagttgaaaaagctgatgctacttttttgtattcttttgaagattctggtgttggtgatgttactggtttcttagcattggataatactaatagattgattgttttgtcattcagaggatctaggtctattgaaaattggatcggtaatttgaactttgaattgaaaggtattgatgatatttgttctggttgtaaaggtcatgctggttttacttcttcttggaggtctgttgctaatactttgactcaacaagttcaaaatgctgttagagaacatcctgattatagagttgtttttactggtcattctttgggtggtgctttggctactgttgctggtgcttctttgagaggtaataaatatgatattgatgttttttcttatggtgctccaagagttggtaatagagcttttgctgaatttttgactgctcaaactggtggtactttgtatagaattactcatactaatgatattgttccaagattgccaccaagagaattgggttattctcattcttctcctgaatattggattacttctggtactttggttcctgttagaaggagagatattgttaaagttgaaggtattgattctactgatggtaataatcaaccaaatactccatctattactgctcatttttggtattttggttctattggtacttgtttgtaa

SEQ ID NO: 6 - protein, 269 aa, synthetic construct
DVSQDLFDQFNLFAQYSAAAYCAKNNRAPAGAIVTCRASICPEVEKADATFLYSFEDSGVGDVTGFLALDNTNRLIVLSFRGSRSIENWIGNLNFELKGIDDICSGCKGHAGFTSSWRSVANTLTQQVQNAVREHPDYRVVFTGHSLGGALATVAGASLRGNKYDIDVFSYGAPRVGNRAFAEFLTAQTGGTLYRITHTNDIVPRLPPREFGYSHSSPEYWITSGTLVPVRRRDIVKVEGIDSTDGNNQPNTPSITAHFWYFGSIGTCL

SEQ ID NO: 7 - DNA, 1077 bp, synthetic construct
  source 1..1077: mol_type = other DNA
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctcttgagaaaagagaggctgaagctgatgtttctcaagatttgtttgatcaatttaatttgtttgctcaatattctgctgcagcttattgtgctaaaaataatagggctcctgctggtgctattgttacttgtagagcttctatttgtcctgaagttgaaaaagctgatgctacttttttgtattcttttgaagattctggtgttggtgatgttactggtttcttagcattggataatactaatagattgattgttttgtcattcagaggatctaggtctattgaaaattggatcggtaatttgaactttgaattgaaaggtattgatgatatttgttctggttgtaaaggtcatgctggttttacttcttcttggaggtctgttgctaatactttgactcaacaagttcaaaatgctgttagagaacatcctgattatagagttgtttttactggtcattctttgggtggtgctttggctactgttgctggtgcttctttgagaggtaataaatatgatattgatgttttttcttatggtgctccaagagttggtaatagagcttttgctgaatttttgactgctcaaactggtggtactttgtatagaattactcatactaatgatattgttccaagattgccaccaagagaatttggttattctcattcttctcctgaatattggattacttctggtactttggttcctgttagaaggagagatattgttaaagttgaaggtattgattctactgatggtaataatcaaccaaatactccatctattactgctcatttttggtattttggttctattggtacttgtttgtaa

SEQ ID NO: 8 - protein, 269 aa, synthetic construct
DVSQDLFDQFNLFAQYSAAAYCAKNNRAPAGAIVTCRASICPEVEKADATFLYSFEDSGVGDVTGFLALDNTNRLIVLSFRGSRSIENWIGNLNWELKGIDDICSGCKGHAGFTSSWRSVANTLTQQVQNAVREHPDYRVVFTGHSLGGALATVAGASLRGNKYDIDVFSYGAPRVGNRAFAEFLTAQTGGTLYRITHTNDIVPRLPPREFGYSHSSPEYWITSGTLVPVRRRDIVKVEGIDSTDGNNQPNTPSITAHFWYFGSIGTCL

SEQ ID NO: 9 - DNA, 1077 bp, synthetic construct
  source 1..1077: mol_type = other DNA
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctcttgagaaaagagaggctgaagctgatgtttctcaagatttgtttgatcaatttaatttgtttgctcaatattctgctgcagcttattgtgctaaaaataatagggctcctgctggtgctattgttacttgtagagcttctatttgtcctgaagttgaaaaagctgatgctacttttttgtattcttttgaagattctggtgttggtgatgttactggttttttggctttggataatactaatagattgattgttttgtcttttagaggatctaggtcaattgaaaactggattggtaatttgaattgggaattgaaaggtattgatgatatttgttctggttgtaaaggtcatgctggttttacttcttcttggaggtctgttgctaatactttgactcaacaagttcaaaatgctgttagagaacatcctgattatagagttgtttttactggtcattctttgggtggtgctttggctactgttgctggtgcttctttgagaggtaataaatatgatattgatgttttttcttatggtgctccaagagttggtaatagagcttttgctgaatttttgactgctcaaactggtggtactttgtatagaattactcatactaatgatattgttccaagattgccaccaagagaatttggttattctcattcttctcctgaatattggattacttctggtactttggttcctgttagaaggagagatattgttaaagttgaaggtattgattctactgatggtaataatcaaccaaatactccatctattactgctcatttttggtattttggttctattggtacttgtttgtaa

SEQ ID NO: 10 - protein, 269 aa, synthetic construct
DVSQDLFDQFNLFAQYSAAAYCAKNNRAPAGAIVTCRASICPEVEKADATFLYSFEDSGVGDVTGFLALDNTNRLIVLSFRGSRSIENWIGNLNYELKGIDDICSGCKGHAGFTSSWRSVANTLTQQVQNAVREHPDYRVVFTGHSLGGALATVAGASLRGNKYDIDVFSYGAPRVGNRAFAEFLTAQTGGTLYRITHTNDIVPRLPPREFGYSHSSPEYWITSGTLVPVRRRDIVKVEGIDSTDGNNQPNTPSITAHFWYFGSIGTCL

SEQ ID NO: 11 - DNA, 1077 bp, synthetic construct
  source 1..1077: mol_type = other DNA
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctcttgagaaaagagaggctgaagctgatgtttctcaagatttgtttgatcaatttaatttgtttgctcaatattctgctgcagcttattgtgctaaaaataatagggctcctgctggtgctattgttacttgtagagcttctatttgtcctgaagttgaaaaagctgatgctacttttttgtattcttttgaagattctggtgttggtgatgttactggttttttggctttggataatactaatagattgattgttttgtcttttagaggatctaggtcaattgaaaactggattggtaatttgaactatgaattgaaaggtattgatgatatttgttctggttgtaaaggtcatgctggttttacttcttcttggaggtctgttgctaatactttgactcaacaagttcaaaatgctgttagagaacatcctgattatagagttgtttttactggtcattctttgggtggtgctttggctactgttgctggtgcttctttgagaggtaataaatatgatattgatgttttttcttatggtgctccaagagttggtaatagagcttttgctgaatttttgactgctcaaactggtggtactttgtatagaattactcatactaatgatattgttccaagattgccaccaagagaatttggttattctcattcttctcctgaatattggattacttctggtactttggttcctgttagaaggagagatattgttaaagttgaaggtattgattctactgatggtaataatcaaccaaatactccatctattactgctcatttttggtattttggttctattggtacttgtttgtaa

SEQ ID NO: 12 - DNA, 738 bp, synthetic construct
  source 1..738: mol_type = other DNA
tggtcagctgaggacaagcataaggaaggtgtgaatagtcacttatggatcgtgaaccgtgccattgatataatgtctaggaatacaactctggttaagcaagatagagttgctcaattgaatgaatggcgtacagagctagagaatggcatctacgctgctgatcatgaaaacccctattacgatgacagtaccttcgcttctcacttttacgatccagacaacggaaagacatatatcccattcgccaagcaagctaaggagactggagctaagtacttcaagttggctggagagtcatacaagaataaagacatgaagcaggccttcttttatcttgggttgtcattgcattatttgggcgatgtcaaccaacctatgcatgccgcatcctttacggacctgtcctatccacagggttttcactccaagtacgagaactttgtcgatactattaaagacaactacaaagttaccgatgggaacggatattggaattggaaaggcaccaaccctgaagaatggattcacggtgcagcagtagttgcaaaacaggactactctggaattgtcaatgacaataccaaagattggtttgtgaaagccgcagtctcccaggaatatgcagataaatggagagctgaagttacacctatgactggtaaacgactaatggatgcccaaagagttactgctggttacattcaattatggttcgacacttacggtgacaggtaa

(19) United States
(12) Patent Application Publication
(10) Pub. No.: US 2026/0209729 A1
(43) Pub. Date: Jul. 23, 2026

(54) Mutated Lipase

(71) Applicant: Wilmar (Shanghai) Biotechnology Research & Development Center Co., LTD., Shanghai (CN)

(72) Inventors: Yaoji Xuan, Shanghai (CN); Guorui Liu, Shanghai (CN); Shengnan Wang, Shanghai (CN); Xiaojun Dai, Shanghai (CN)

(21) Appl. No.: 19/140,228

(22) PCT Filed: Dec. 14, 2023
(86) PCT No.: PCT/CN2023/138703

§ 371 Date: Jun. 17, 2025

(30) Foreign Application Priority Data
Dec. 21, 2022 (CN) 202211647293.3

(51) Int. Cl. C12N 9/20; C12N 1/20; C12N 15/81; C12P 7/649; C12R 1/84

(52) U.S. Cl. CPC C12N 9/20; C12N 1/20; C12N 15/815; C12P 7/649; C12R 2001/84; C12Y 301/01003

(57) ABSTRACT

The present invention relates to a mutated lipase. The present invention further relates to a gene encoding the lipase, and a vector and a host cell comprising the gene. In addition, the present invention further relates to use of the lipase.

### TECHNICAL FIELD

The present invention relates to the field of biotechnology, and more specifically to a mutant lipase. It also relates to a gene encoding the enzyme, and a vector and a host cell comprising the gene. Moreover, it relates to the use of the enzyme.

### BACKGROUND

As an important petroleum refining product, diesel occupies a high share in the composition of the fuel of various countries and has become an important power fuel. With the acceleration of the dieselization trend of vehicles worldwide, the demand for diesel will become larger and larger in the future. The increasing depletion of petroleum resources and the improvement of people's environmental awareness have greatly promoted the development of diesel which replaces fuels in various countries around the world. Especially since the 1990s, biodiesel has attracted attention from various countries for its superior environmental protection performance. At present, the annual production of new cars in the world is about 50 million, and the number of cars in the world is about 750 million (including motorcycles). With the rapid development of the automobile industry, the use of gasoline and diesel has increased with the increase in the number of cars, which has also brought about problems such as automobile exhaust pollution.

It is well-known that diesel molecules are composed of about 15 carbon chains. Studies have found that plant oil molecules are generally composed of 14 to 18 carbon chains, which is similar to the number of carbon atoms in diesel molecules. Therefore, biodiesel is a new type of fuel processed from renewable plant oil such as rapeseed. Compared with conventional diesel, biodiesel has the following incomparable properties: (1) It has excellent environmental protection characteristics. The use of biodiesel can reduce air toxicity by 90% and reduce the cancer rate by 94%; carbon monoxide emissions are reduced by about 10% compared with diesel. (2) It has good low-temperature engine starting performance. (3) It has good lubrication performance. (4) It has good safety performance. (5) It has good fuel performance. (6) It has renewable properties. The carbon dioxide emitted when biodiesel is burned is much less than the carbon dioxide absorbed during the growth of the plant, thereby improving the global warming caused by carbon dioxide emissions, a major environmental problem that is harmful to mankind. Therefore, biodiesel is a truly green diesel.

Currently, United States and some countries and regions in Europe and Asia have begun to establish commercial biodiesel production bases and widely use biodiesel as an alternative fuel. This is mainly because the United States has soybeans, Europe has rapeseed, and Asia has palm oil. The sustainable development of these agricultural industries requires finding ways to consume large quantities of vegetable oil other than food and chemical industries, guaranteeing the consumption of biodiesel in legal form, and promoting the development of the biodiesel industry with subsidies.

At present, biodiesel is mainly produced by chemical methods, that is, animal and vegetable oil and low-carbon alcohols such as methanol or ethanol are subjected to transesterification reaction under acid or alkaline catalysts and high temperature (230-250° C.) to generate corresponding fatty acid methyl esters or ethyl esters, which are then washed and dried to obtain biodiesel. Methanol or ethanol can be recycled in the production process, and the production equipment is the same as general oil production equipment. About 10% of glycerol can be produced as a by-product during the production process.

The main problem of biodiesel at present is its high cost. According to statistics, 75% of the cost of biodiesel production is the cost of raw materials. Therefore, the use of cheap raw materials and improving conversion to reduce costs are the key to whether biodiesel can be practical. The United States has begun to study plants with high oil content through genetic engineering methods. Japan uses industrial waste oil and waste frying oil. Europe grows oil-rich crops on land that is not suitable for growing food. Asia uses waste oil and oil processing by-products for production.

However, chemical synthesis of biodiesel has the following disadvantages: the process is complex, alcohol must be in excess, and the subsequent process must have a corresponding alcohol recovery device, which consumes a lot of energy; the color is dark because the unsaturated fatty acids in the fat are easily deteriorated at high temperatures; the esterification products are difficult to recover and the cost is high; and waste alkali liquid is discharged during the production process.

To solve the above problems, people began to study the synthesis of biodiesel by bioenzyme, that is, using animal fat and low-carbon alcohols to undergo transesterification reactions with lipases to prepare the corresponding fatty acid methyl esters and ethyl esters. Enzymatic synthesis of biodiesel has the advantages of mild conditions, small alcohol dosage, and no pollution emissions. However, the main problems are: poor high temperature stability of enzymes, certain toxicity of short-chain alcohols to enzymes, and short life of enzymes. The by-products glycerol and water are difficult to recover, which not only inhibits product formation, but also glycerol is toxic to immobilized enzymes, shortening the life of immobilized enzymes.

The sequence of Thermomyces dupontii lipase (TDL) was published in 2012. The protein is 291 amino acids long, with a 22-amino acid signal peptide and a mature peptide of 269 amino acids. It has a typical lipase triplet catalytic domain structure of Ser168-Asp223-His280. TDL has a high preference for hydrolyzing C8 triglycerides at 50° C. and pH 9.0. The conversion rate of biodiesel produced from waste cooking oil (WCO) is 91.6%.

In the applicant's patent application CN 2016112256785, the inventor obtained a TDL mutant EP14. At a protein concentration of 20 mg/ml, an addition amount of 0.5%, and a reaction time of 48 hours, its biodiesel conversion rate can reach 93%. However, it does not meet the biodiesel standard, that is, the conversion rate of 96.5%.

Therefore, there is still a need in the art for new lipases with high conversion rates for biodiesel conversion.

### SUMMARY OF THE INVENTION

In the present invention, the inventors used the amino acid sequence of EP14 in CN 2016112256785 as the starting sequence, introduced seven mutation sites D27R, G38A, D96E, D111A, G163K, D254S and A256T by site-directed mutagenesis, and obtained the mutant TDLm2. TDLm2 was incubated at 35° C. and 80% methanol for 6 hours, and the residual enzyme activity was increased from 7.7% of EP14 to 72%.

A biodiesel reaction was carried out using fatty matter as a substrate, with a protein concentration of 20 mg/ml, an addition amount of 0.45%, and a reaction time of 24 hours. The conversion rate could reach 94%.

Based on TDLm2, three mutation sites L86I, 193L and M95F were introduced by site-directed mutagenesis to obtain mutant LM. Based on TDLm2, four mutation sites L86I, 193L, M95F and L211F were introduced to obtain mutant LMB.

A biodiesel reaction was carried out using fatty matter as a substrate, with a protein concentration of 20 mg/ml and an addition amount of 0.45%. The conversion rate could reach 94% after 8 hours of LM reaction. After 24 hours of reaction, the conversion rate could reach 96.5%. After 8 hours of reaction, the conversion rate of LMB reaction could reach 95%. After 24 hours of reaction, the conversion rate could reach 96.5%.

Based on LMB, the point mutation F95W was introduced to obtain the mutant LMBW; the point mutation F95Y was introduced to obtain the mutant LMBY.

A biodiesel reaction was carried out using fatty matter as a substrate, with a protein concentration of 20 mg/ml and an addition amount of 0.45%. The conversion rate could reach 92% after 8 hours of LMBW reaction. After 24 hours of reaction, the conversion rate could reach 96.6%. After 8 hours of LMBY reaction, the conversion rate could reach 96%. After 24 hours of reaction, the conversion rate could reach 96.6%.

The biodiesel conversion rates of the mutants TDLm2, LM, LMB, LMBW and LMBY obtained by the invention are greatly improved, and the amount of enzyme used and the reaction time can be reduced, thereby reducing the production cost of biodiesel.

Specifically, the present invention relates to the following aspects:

In one aspect, the present invention relates to a lipase comprising an amino acid sequence having a mutation relative to SEQ ID NO: 1 at one or more amino acid positions corresponding to positions 27, 38, 96, 111, 163, 254 and 256 of SEQ ID NO: 1. In one embodiment, the mutation is one or more of D27R, G38A, D96E, D111A, G163K, D254S and A256T.

In one embodiment, the lipase of the present invention further comprises an additional mutation at one or more amino acid positions corresponding to positions 86, 93, 95 and 211 of SEQ ID NO: 1. In one embodiment, the additional mutation is a mutation at position 95 of SEQ ID NO: 1, and one or more of L86I, 193L and L211F. In one embodiment, the additional mutation is one or more of L86I, 193L and M95F; L86I, 193L, M95F and L211F; L86I, 193L, M95W and L211F; or L86I, 193L, M95Y and L211F.

In one embodiment, the lipase of the present invention comprises an amino acid sequence selected from the group consisting of SEQ ID NO: 2, 4, 6, 8 and 10, or a sequence having at least 90%, or at least 95% or at least 99% sequence identity thereto.

As used herein, “percent identity” refers to the degree to which two optimally aligned DNA or protein segments remain unchanged throughout a comparison window of components such as nucleotide sequences or amino acid sequences. The “identity fraction” for the comparison segments of a test sequence and a reference sequence is the number of identical components shared by the two comparison segment sequences through the comparison window divided by the total number of sequence components in the reference segment, the total number being the smaller of the complete test sequence or the complete reference sequence. “Percent identity” (“identity %”) is the identity fraction multiplied by 100.

In another aspect, the present invention relates to a method for preparing a lipase mutant, comprising introducing one or more mutations at amino acid positions of a starting lipase corresponding to positions 27, 38, 96, 111, 163, 254 and 256 of SEQ ID NO: 1. In one embodiment, the mutation is one or more of D27R, G38A, D96E, D111A, G163K, D254S and A256T. In one embodiment, the method further comprises introducing one or more mutations at amino acid positions of a starting lipase corresponding to positions 86, 93, 95 and 211 of SEQ ID NO: 1. In one embodiment, the mutation is one or more of L86I, 193L and M95F; L86I, 193L, M95F and L211F; L86I, 193L, M95W and L211F; or L86I, 193L, M95Y and L211F. In one embodiment, the sequence of the starting lipase is as shown in SEQ ID NO: 1.

In another aspect, the present invention relates to a nucleic acid molecule selected from the group consisting of: (a) a nucleotide sequence encoding the above-mentioned lipase, or a sequence having at least 90%, at least 95% or at least 99% sequence identity thereto; and (b) a nucleotide sequence complementary to the nucleotide sequence described in (a), which may be partially or completely complementary. In one embodiment, the nucleic acid molecule comprises a nucleotide sequence selected from SEQ ID NO: 3, 5, 7, 9 and 11, or a sequence having at least 90%, at least 95% or at least 99% sequence identity thereto.

The skilled in the art will understand, due to the degeneracy of the genetic code, a variety of different nucleotide sequences can encode the same enzyme. In addition, it will be appreciated that the skilled in the art can use routine techniques to replace nucleotides that do not affect the enzymatic activity encoded by the nucleotide sequence of the present invention, which can reflect the codon bias of any specific host organism used to express the enzyme of the present invention.

The present invention also provides a vector comprising the nucleic acid molecule, and a host cell comprising the nucleic acid molecule or the vector.

“Vector” refers to an extrachromosomal element that usually carries a gene that is not a part of the central metabolism of a cell, and is often in the form of a circular double-stranded DNA molecule. Such elements may be autonomously replicating sequences, genomic integration sequences, phage or nucleotide sequences, linear or circular single- or double-stranded DNAs or RNAs from any source, in which many of the nucleotide sequences have been spliced or recombined into specific constructs that are capable of introducing the promoter fragments and DNA sequences of the selected gene products together with appropriate 3′ untranslated sequences into a cell.

Genes and gene products encoding the lipases of the invention can be expressed in heterologous host cells, such as bacterial cells, fungal cells, such as yeast cells, mammalian cells, insect cells and plant cells. Heterologous host cells for expressing the nucleic acid molecules of the invention can be microbial hosts in fungal or bacterial families and grow over a wide range of temperature, pH, and solvent tolerance. For example, it is contemplated that any bacteria, yeast, and filamentous fungi may be suitable hosts for expression of the nucleic acid molecules of the invention. Examples of host strains include, but are not limited to, bacterial, fungal or yeast species such as Pichia, Aspergillus, Trichoderma, Saccharomyces, Phaffia, Kluyveromyces, Yarrowia, Candida, Hansenula, Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella and Myxococcus species. In one embodiment, the host cell is a fungal cell. In one embodiment, the host cell is Pichia pastoris.

Vectors useful for transforming the host cells described above are well known in the art. Typically, vectors contain sequences that direct transcription and translation of the related genes, selectable markers, and sequences that allow autonomous replication or chromosomal integration. A suitable vector contains a 5′ region of the gene that controls transcription initiation and a 3′ region of the DNA fragment that controls transcription termination.

In one aspect, the present invention also relates to a method for producing a lipase, comprising expressing a nucleic acid molecule encoding the lipase of the present invention in a host cell, and recovering the resulting polypeptide.

A variety of culture methods can be used to prepare the enzymes of the invention. For example, large-scale production of specific gene products from recombinant microbial hosts can be performed by batch, fed-batch, and continuous culture methods.

Batch and fed-batch culture methods are commonly used and well known in the art, and examples can be found in: Thomas D. Brock in Biotechnology: A Textbook of Industrial Microbiology, Second Edition, Sinauer Associates, Inc., Sunderland, MA (1989)), and Deshpande, Mukund V., (Appl. Biochem. Biotechnol., 36:227-234 (1992).

Commercial production of the enzymes of the invention can also be carried out by continuous culture. Continuous culture is an open system in which conditioned media are continuously added to the bioreactor and equal amounts of conditioned media are simultaneously removed for processing. Continuous culture generally maintains cells at a constant high liquid density where the cells are primarily in the logarithmic phase of growth. Alternatively, continuous culture can be performed with immobilized cells, wherein carbon and nutrients are continuously added and valuable products, by-products or waste products are continuously removed from the cell pellet. Cell immobilization can be performed using a wide range of solid supports consisting of natural and/or synthetic materials.

Recovery of the desired enzyme from batch fermentation, fed-batch fermentation, or continuous culture can be accomplished by any method known to those skilled in the art. For example, when an enzyme is produced intracellularly, the cell slurry is separated from the culture medium by centrifugation or membrane filtration, optionally washed with water or an aqueous buffer of the desired pH. Then the cell slurry in the aqueous buffer of the desired pH is suspended to be homogenized to produce a cell extract containing the required enzyme.

The present invention also relates to a composition comprising the lipase of the invention, or a fermentation broth, fermentation supernatant and/or fermentation concentrate of the host cell of the invention. The enzyme composition of the present invention may be in any form suitable for use, for example, crude fermentation broth with or without cell removal, cell lysate with or without cell debris, semi-purified or purified enzyme composition, or host cells as a source of enzymes. The enzyme composition may be a dry powder or granule, a dust-free granule, a liquid, a stabilized liquid or a stabilized protected enzyme. The liquid enzyme composition can be stabilized according to established processes, for example by adding stabilizers such as sugars, sugar alcohols or other polyols, and/or lactic acid or other organic acids. The enzyme composition of the present invention may also include other lipases, for example: Lipex Evity 200L, Lipozyme 435, Lipase A “Amano” 6, Lipase AY “Amano” 30SD, Lipase G “Amano” 50, Lipase R “Amano”, Lipase DF “Amano” 15, Lipase MER “Amano”, Newlase F.

The present invention further relates to the fermentation broth, fermentation supernatant or fermentation concentrate of the host cell of the present invention.

The present invention also relates to use of the lipase of the present invention in converting fatty matter into biodiesel. When the lipase of the present invention is used for biodiesel conversion, compared with the lipase of the prior art, the amount of enzyme used and the reaction time can be reduced, thereby reducing the production cost of biodiesel.

The present invention also relates to a method for preparing biodiesel, comprising incubating raw material oil, a low-carbon alcohol, and the lipase or the composition or the fermentation broth, the fermentation supernatant, and/or the fermentation concentrate of the host cell of the present invention together to produce corresponding fatty acid ester. In one embodiment, the raw material oil is selected from oil of oil crops, wild oil plants and aquatic plants such as engineered microalgae, animal fat and waste cooking oil, and the low-carbon alcohol is selected from methanol and ethanol.

### BRIEF DESCRIPTION OF THE DRAWINGS

FIG. 1 shows the results of the comparison of methanol tolerance between TDLm2 of the present invention and EP14 of the prior art.

FIG. 2 shows the results of measuring the concentrations of PTDLm2, PLM, PLMB, PLMBW and PLMBY in the fermentation broth using a modified Bradford protein concentration determination kit.

### DETAILED DESCRIPTION

### Experimental Materials

### 1. Experimental Strains and Plasmids

Strain: Pichia pastoris M316 (prepared by the applicant's previous work, deposit number CGMCC19221, see patent application number WO2020135763A1).

Plasmid: pET-pAOm-plc plasmid, synthesized by Shanghai Sangon Biotech Co., Ltd. The synthesis steps were as follows: the pAO815 vector sequence was prepared, which carried the encoding sequence of the PLC mutant sequence in the patent CN201680072289.5 “Highly efficient zinc-independent phospholipase C mutant” (SEQ ID NO: 7 of CN201680072289.5, Xaa is His) in the expression cassette, and was ligated into the pET-28C vector.

The encoding sequence of the PLC mutant sequence in the above patent CN201680072289.5 “Highly efficient zinc-independent phospholipase C mutant” is:

(SEQ ID NO: 12)
tggtcagctgaggacaagcataaggaaggtgtgaatagtcacttatggatcgtgaaccgtgccattgatataatgtcta
ggaatacaactctggttaagcaagatagagttgctcaattgaatgaatggcgtacagagctagagaatggcatctacg
ctgctgatcatgaaaacccctattacgatgacagtaccttcgcttctcacttttacgatccagacaacggaaagacatat
atcccattcgccaagcaagctaaggagactggagctaagtacttcaagttggctggagagtcatacaagaataaag
acatgaagcaggccttcttttatcttgggttgtcattgcattatttgggcgatgtcaaccaacctatgcatgccgcatccttta
cggacctgtcctatccacagggttttcactccaagtacgagaactttgtcgatactattaaagacaactacaaagttacc
gatgggaacggatattggaattggaaaggcaccaaccctgaagaatggattcacggtgcagcagtagttgcaaaac
aggactactctggaattgtcaatgacaataccaaagattggtttgtgaaagccgcagtctcccaggaatatgcagata
aatggagagctgaagttacacctatgactggtaaacgactaatggatgcccaaagagttactgctggttacattcaatt
atggttcgacacttacggtgacaggtaa.

### 2. Culture Media and Solutions

YPD liquid medium: 1% yeast extract, 2% peptone, 2% glucose.

YPD solid medium: 2% agar was added into LB liquid medium.

BMMY-olive oil screening medium: Component A: 1% yeast extract, 2% peptone, 1.34% yeast nitrogen base (YNB) containing ammonium sulfate but not amino acids, 1% glycerol, 4×10⁻⁵% D-biotin (added after sterilization), 0.1M citric acid-sodium citrate buffer pH 6.6, 2% agar. Component B: Olive oil substrate solution: 150 ml of 4% PVA solution was taken, added with 50 ml of olive oil, emulsified with a high-speed homogenizer at 8000 rpm for 3 min, paused for 1 min and then emulsified for 3 min to prepare the substrate solution. 100 ml of sterilized component A was mixed with 12 ml of component B, added with 1 ml of 0.1% rhodamine B and 2% methanol.

BMGY liquid medium: 1% yeast extract, 2% peptone, 1.34% yeast nitrogen base (YNB) containing ammonium sulfate but not amino acids, 1% glycerol, 4×10⁻⁵% D-biotin, 0.1M citric acid-sodium citrate buffer pH 6.6.

BMMY liquid medium: 1% yeast extract, 2% peptone, 1.34% yeast nitrogen base (YNB) containing ammonium sulfate but not amino acids, 2% methanol, 4×10⁵% D-biotin, 0.1M citric acid-sodium citrate buffer pH 6.6.

The modified Bradford protein concentration determination kit was purchased from Shanghai Sangon Biotech Co., Ltd.

PCR enzyme: PrimeSTAR@HS DNA Polymerase (purchased from TakaraBio (Dalian) Co., Ltd.).

### Example 1: Expression of Mutant TDLm2 in Pichia pastoris and Preparation of Enzyme Solution

The amino acid sequence of the mature peptide of EP14 in CN 2016112256785 is as follows:

(EP14, SEQ ID NO: 1)
DVSQDLFDQFNLFAQYSAAAYCAKNNDAPAGAIVTCRGSICPEVEKADATFLYSFED
SGVGDVTGFLALDNTNRLIVLSFRGSRSLENWIGNINMDLKGIDDICSGCKGHDGFT
SSWRSVANTLTQQVQNAVREHPDYRVVFTGHSLGGALATVAGASLRGNGYDIDVFS
YGAPRVGNRAFAEFLTAQTGGTLYRITHTNDIVPRLPPReLGYSHSSPEYWITSGTLV
PVRRRDIVKVEGIDSTDGNNQPNTPDIAAHFWYFGSIGTCL.

Seven mutation sites, D27R, G38A, D96E, D111A, G163K, D254S and A256T, were introduced into SEQ ID NO: 1 to obtain the amino acid sequence

(TDLm2, SEQ ID NO: 2)
DVSQDLFDQFNLFAQYSAAAYCAKNNRAPAGAIVTCRASICPEVEKADATFLYSFED
SGVGDVTGFLALDNTNRLIVLSFRGSRSLENWIGNINMELKGIDDICSGCKGHAGFT
SSWRSVANTLTQQVQNAVREHPDYRVVFTGHSLGGALATVAGASLRGNKYDIDVFS
YGAPRVGNRAFAEFLTAQTGGTLYRITHTNDIVPRLPPRELGYSHSSPEYWITSGTLV
PVRRRDIVKVEGIDSTDGNNQPNTPSITAHFWYFGSIGTCL.

The DNA sequence was designed based on the codon preference of Pichia pastoris and the prepropeptide sequence of a-factor (derived from the DNA sequence of the commercial vector pPIC9K) was added. The following DNA encoding sequence was obtained:

(encoding sequence of TDLm2, SEQ ID NO: 3)
ATGAGATTTCCTTCAATTTTTACTGCAGTTTTATTCGCAGCATCCTCCGCATTAGCT
GCTCCAGTCAACACTACAACAGAAGATGAAACGGCACAAATTCCGGCTGAAGCT
GTCATCGGTTACTCAGATTTAGAAGGGGATTTCGATGTTGCTGTTTTGCCATTTTC
CAACAGCACAAATAACGGGTTATTGTTTATAAATACTACTATTGCCAGCATTGCTG
CTAAAGAAGAAGGGGTATCTCTTGAGAAAAGAGAGGCTGAAGCTGATGTTTCTC
AAGATTTGTTTGATCAATTTAATTTGTTTGCTCAATATTCTGCTGCAGCTTATTGTG
CTAAAAATAATAGGGCTCCTGCTGGTGCTATTGTTACTTGTAGAGCTTCTATTTGT
CCTGAAGTTGAAAAAGCTGATGCTACTTTTTTGTATTCTTTTGAAGATTCTGGTGT
TGGTGATGTTACTGGTTTTTTGGCTTTGGATAATACTAATAGATTGATTGTTTTGTC
TTTTAGAGGTTCTAGGTCATTAGAAAATTGGATTGGTAATATTAACATGGAATTAAA
AGGTATTGATGATATTTGTTCTGGTTGTAAAGGTCATGCTGGTTTTACTTCTTCTTG
GAGGTCTGTTGCTAATACTTTGACTCAACAAGTTCAAAATGCTGTTAGAGAACATC
CTGATTATAGAGTTGTTTTTACTGGTCATTCTTTGGGTGGTGCTTTGGCTACTGTT
GCTGGTGCTTCTTTGAGAGGTAATAAATATGATATTGATGTTTTTTCTTATGGTGCT
CCAAGAGTTGGTAATAGAGCTTTTGCTGAATTTTTGACTGCTCAAACTGGTGGTA
CTTTGTATAGAATTACTCATACTAATGATATTGTTCCAAGATTGCCACCAAGAGAAT
TGGGTTATTCTCATTCTTCTCCTGAATATTGGATTACTTCTGGTACTTTGGTTCCTG
TTAGAAGGAGAGATATTGTTAAAGTTGAAGGTATTGATTCTACTGATGGTAATAATC
AACCAAATACTCCATCTATTACTGCTCATTTTTGGTATTTTGGTTCTATTGGTACTT
GTTTGtaa.

The DNA sequence was sent to Suzhou Genewiz Biotechnology Co., Ltd. for full gene synthesis and was cloned into pET-pAOm-plc vector through the Sacll and EcoRI sites to obtain the plasmid p-TDLm2.

p-TDLm2 was linearized with Sall, and competent cells of Pichia pastoris GS115 were prepared by LiAC method. Then, the linearized p-TDLm2 fragment was transformed into GS115 competent cells by electroporation. The transformants were coated on a MGYS plate and cultured at 30° C. for 3 days. A large number of single clones on the plate were picked on a BMMY-olive oil screening plate, and positive clones with the best activity performance were picked out and named PTDLm2.

PTDLm2 and EP14 strains (EP14 strain was obtained with the encoding sequence of EP14 enzyme (SEQ ID NO: 6 in patent application CN 201611225678.5) by the same construction method as PTDLm2 strain) were first activated in liquid YPD, inoculated in BMGY, cultured overnight at 30° C., 220 rpm, and then transferred to BMGY medium (the initial OD600 was 6), and initially induced with 2% methanol. 1% methanol was added after 24 h and 32 h respectively, and 1% methanol was added after 48 h and 56 h respectively. After 72 h, the sample was taken, and concentrated with an ultrafiltration tube with a molecular weight cutoff of 10 KD to make the protein concentration reach 30 mg/ml.

The enzyme solutions of pTDLm2 and EP14 were adjusted to 30 mg/ml. The residual enzyme activity was measured after incubated at 35° C. for 6 hours at a methanol concentration of 80%. The experiments were set up in 2 parallels and repeated twice. The enzyme activity of the untreated enzyme solution was used as a control, which had the relative enzyme activity recorded as 100%. The enzyme activity determination method was as follows:

150 ml of 4% PVA solution was taken, added with 50 ml of olive oil, and emulsified with a high-speed homogenizer at 8000 rpm for 3 minutes. After a 1-minute pause, it was emulsified for another 3 minutes to prepare the substrate solution (this solution must be prepared and used immediately).

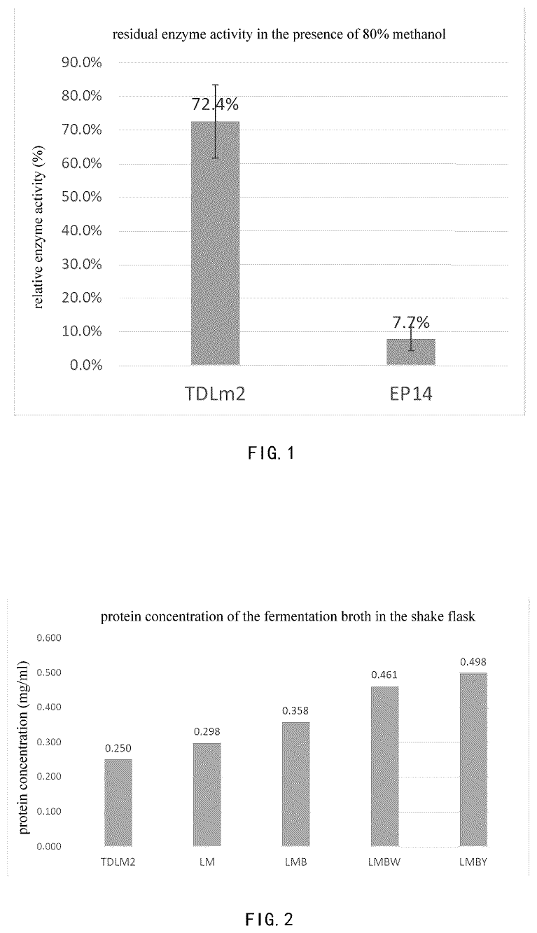

The residual enzyme activities of pTDLm2 and EP14 after incubation at 35° C. for 6 hours in the presence of 80% methanol are shown in FIG. 1.

### Example 2: Test of TDLm2 Biodiesel Reaction

### Biodiesel Preparation Method:

1. Raw materials: Fatty matter (50%-60% palmitic acid, 40%-50% palmitic acid methyl ester, specifically, palmitic acid (purchased from Shanghai Aladdin Biochemical Technology Co., Ltd.) and palmitic acid methyl ester (purchased from Shanghai Aladdin Biochemical Technology Co., Ltd.) were mixed at a ratio of 5-6:4-5. In the following examples, the specific Fatty matter used was obtained by mixing in a ratio of 6:4. Methanol was purchased from Sinopharm Group.

### Reaction System and Reaction Conditions:

Initial reaction system: Fatty matter 1 ml+methanol 150 ul+enzyme solution 4.5 μl (protein concentration was 20 mg/ml)+water 30 ul

After methanol and Fatty matter were evenly mixed, enzyme solution and water were added. The sample was placed in a metal bath for incubation at 35° C. and 1000 rpm. After 2.5 hours of reaction, 100 ul of methanol was added. After 4.5 hours of reaction, 50 ul of methanol was added. After 8 hours and 24 hours of reaction, the acid values were measured respectively, and the free fatty acid (FFA) % was calculated.
2. FFA test method: The oil sample must be liquid and evenly mixed before measurement.
3. Approximately 0.2 g of oil sample were weighed, accurated to 0.001 g, and placed in a 50 mL centrifuge tube.
4. 10 mL of neutral isopropanol was added (several drops of 1% phenolphthalein indicator were added). The oil sample was fully dissolved by shaking (heated if necessary).
5. 0.05 mol/L potassium hydroxide standard solution was used to titrate while shaken until pink appeared. If the color did not fade within 30 seconds, it was the titration endpoint.
6. The volume (V) of potassium hydroxide consumed in the titration was recorded and the result was calculated.

### Representation of Analysis Results

$$Acid value = V \times c \times 5 6.1 / m$$

Wherein:
V—volume of potassium hydroxide solution used, ml;
c—the exact concentration of potassium hydroxide standard solution used, mol/L;
M—sample mass, g;
56.1—molar mass of potassium hydroxide, g/mol.

$$FFA \% = acid value / 2 \times 100 \%$$

The results of biodiesel production of TDLm2 and EP14 are shown in the following table:

| Reaction | FFA%/conversion | FFA%/conversion |
|---|---|---|
| time | rate (TDLm2) | rate (EP14) |
| 8 h | 15%/85% | 44%/56% |
| 24 h | 6%/94% | 42%/58% |

It can be seen that TDLm2 of the present invention leads to a significantly improved biodiesel conversion rate compared to EP14.

### Example 3: Expression of Mutants LM and LMB in Pichia pastoris and Preparation of Enzyme Solution

The amino acid sequence of the mature peptide of TDLm2 was introduced with three mutation sites, L86I, 193L and M95F to obtain the following amino acid sequence:

(LM, SEQ ID NO: 4)
DVSQDLFDQFNLFAQYSAAAYCAKNNRAPAGAIVTCRASICPEVEKADATFLYSFED
SGVGDVTGFLALDNTNRLIVLSFRGSRSIENWIGNLNFELKGIDDICSGCKGHAGFT
SSWRSVANTLTQQVQNAVREHPDYRVVFTGHSLGGALATVAGASLRGNKYDIDVFS
YGAPRVGNRAFAEFLTAQTGGTLYRITHTNDIVPRLPPRELGYSHSSPEYWITSGTLV
PVRRRDIVKVEGIDSTDGNNQPNTPSITAHFWYFGSIGTCL.

The DNA sequence was designed based on the codon preference of Pichia pastoris and the prepropeptide sequence of a-factor (derived from the DNA sequence of the commercial vector pPIC9K) was added. The following DNA sequence was obtained:

(encoding sequence of LM, SEQ ID NO: 5)
ATGAGATTTCCTTCAATTTTTACTGCAGTTTTATTCGCAGCATCCTCCGCATTAGCT
GCTCCAGTCAACACTACAACAGAAGATGAAACGGCACAAATTCCGGCTGAAGCT
GTCATCGGTTACTCAGATTTAGAAGGGGATTTCGATGTTGCTGTTTTGCCATTTTC
CAACAGCACAAATAACGGGTTATTGTTTATAAATACTACTATTGCCAGCATTGCTG
CTAAAGAAGAAGGGGTATCTCTTGAGAAAAGAGAGGCTGAAGCTGATGTTTCTC
AAGATTTGTTTGATCAATTTAATTTGTTTGCTCAATATTCTGCTGCAGCTTATTGTG
CTAAAAATAATAGGGCTCCTGCTGGTGCTATTGTTACTTGTAGAGCTTCTATTTGT
CCTGAAGTTGAAAAAGCTGATGCTACTTTTTTGTATTCTTTTGAAGATTCTGGTGT
TGGTGATGTTACTGGTTTCTTAGCATTGGATAATACTAATAGATTGATTGTTTTGTC
ATTCAGAGGATCTAGGTCTATTGAAAATTGGATCGGTAATTTGAACTTTGAATTGA
AAGGTATTGATGATATTTGTTCTGGTTGTAAAGGTCATGCTGGTTTTACTTCTTCTT
GGAGGTCTGTTGCTAATACTTTGACTCAACAAGTTCAAAATGCTGTTAGAGAACA
TCCTGATTATAGAGTTGTTTTTACTGGTCATTCTTTGGGTGGTGCTTTGGCTACTG
TTGCTGGTGCTTCTTTGAGAGGTAATAAATATGATATTGATGTTTTTTCTTATGGTG
CTCCAAGAGTTGGTAATAGAGCTTTTGCTGAATTTTTGACTGCTCAAACTGGTGG
TACTTTGTATAGAATTACTCATACTAATGATATTGTTCCAAGATTGCCACCAAGAGA
ATTGGGTTATTCTCATTCTTCTCCTGAATATTGGATTACTTCTGGTACTTTGGTTCC
TGTTAGAAGGAGAGATATTGTTAAAGTTGAAGGTATTGATTCTACTGATGGTAATAA
TCAACCAAATACTCCATCTATTACTGCTCATTTTTGGTATTTTGGTTCTATTGGTAC
TTGTTTGtaa.

The DNA sequence was sent to Suzhou Genewiz Biotechnology Co., Ltd. for full gene synthesis and was cloned into pET-pAOm-plc vector through the Sacll and EcoRI sites to obtain the plasmid p-LM.

The amino acid sequence of the mature peptide of TDLm2 was introduced with four mutation sites, L86I, 193L, M95F and L211F to obtain the following amino acid sequence:

(LMB, SEQ ID NO: 6)
DVSQDLFDQFNLFAQYSAAAYCAKNNRAPAGAIVTCRASICPEVEKADATFLYSFED
SGVGDVTGFLALDNTNRLIVLSFRGSRSIENWIGNLNFELKGIDDICSGCKGHAGFT
SSWRSVANTLTQQVQNAVREHPDYRVVFTGHSLGGALATVAGASLRGNKYDIDVFS
YGAPRVGNRAFAEFLTAQTGGTLYRITHTNDIVPRLPPREFGYSHSSPEYWITSGTLV
PVRRRDIVKVEGIDSTDGNNQPNTPSITAHFWYFGSIGTCL

The DNA sequence was designed based on the codon preference of Pichia pastoris and the prepropeptide sequence of a-factor (derived from the DNA sequence of the commercial vector pPIC9K) was added. The following DNA sequence was obtained:

(encoding sequence of LMB, SEQ ID NO: 7)
ATGAGATTTCCTTCAATTTTTACTGCAGTTTTATTCGCAGCATCCTCCGCATTAGCT
GCTCCAGTCAACACTACAACAGAAGATGAAACGGCACAAATTCCGGCTGAAGCT
GTCATCGGTTACTCAGATTTAGAAGGGGATTTCGATGTTGCTGTTTTGCCATTTTC
CAACAGCACAAATAACGGGTTATTGTTTATAAATACTACTATTGCCAGCATTGCTG
CTAAAGAAGAAGGGGTATCTCTTGAGAAAAGAGAGGCTGAAGCTGATGTTTCTC
AAGATTTGTTTGATCAATTTAATTTGTTTGCTCAATATTCTGCTGCAGCTTATTGTG
CTAAAAATAATAGGGCTCCTGCTGGTGCTATTGTTACTTGTAGAGCTTCTATTTGT
CCTGAAGTTGAAAAAGCTGATGCTACTTTTTTGTATTCTTTTGAAGATTCTGGTGT
TGGTGATGTTACTGGTTTCTTAGCATTGGATAATACTAATAGATTGATTGTTTTGTC
ATTCAGAGGATCTAGGTCTATTGAAAATTGGATCGGTAATTTGAACTTTGAATTGA
AAGGTATTGATGATATTTGTTCTGGTTGTAAAGGTCATGCTGGTTTTACTTCTTCTT
GGAGGTCTGTTGCTAATACTTTGACTCAACAAGTTCAAAATGCTGTTAGAGAACA
TCCTGATTATAGAGTTGTTTTTACTGGTCATTCTTTGGGTGGTGCTTTGGCTACTG
TTGCTGGTGCTTCTTTGAGAGGTAATAAATATGATATTGATGTTTTTTCTTATGGTG
CTCCAAGAGTTGGTAATAGAGCTTTTGCTGAATTTTTGACTGCTCAAACTGGTGG
TACTTTGTATAGAATTACTCATACTAATGATATTGTTCCAAGATTGCCACCAAGAGA
ATTTGGTTATTCTCATTCTTCTCCTGAATATTGGATTACTTCTGGTACTTTGGTTCC
TGTTAGAAGGAGAGATATTGTTAAAGTTGAAGGTATTGATTCTACTGATGGTAATAA
TCAACCAAATACTCCATCTATTACTGCTCATTTTTGGTATTTTGGTTCTATTGGTAC
TTGTTTGtaa.

The DNA sequence was sent to Suzhou Genewiz Biotechnology Co., Ltd. for full gene synthesis and was cloned into pET-pAOm-plc vector through the Sacll and EcoRI sites to obtain the plasmid p-LMB.

p-LM and p-LMB were linearized with Sall, and competent cells of Pichia pastoris GS115 were prepared by the LiAC method. The linearized p-LM and p-LMB fragments were then transformed into GS115 competent cells by electroporation. The transformants were coated on a MGYS plate and cultured at 30° C. for 3 days. A large number of single clones on the plate were picked on a BMMY-olive oil screening plate, and positive clones with the best activity performance were picked out and named PLM and PLMB.

PLM and PLMB strains were first activated in liquid YPD, inoculated in BMGY, cultured overnight at 30° C., 220 rpm, and then transferred to BMGY medium (the initial OD600 was 6), and initially induced with 2% methanol. 1% methanol was added after 24 h and 32 h respectively, and 1% methanol was added after 48 h and 56 h respectively. After 72 h, the sample was concentrated with an ultrafiltration tube with a molecular weight cutoff of 10 KD to make the protein concentration reach 30 mg/ml.

### Example 4: Biodiesel Reaction Tests of LM and LMB

The biodiesel preparation method is as described in Example 2.

The results of biodiesel production of LM and LMB are shown in the following table.

| Reaction | FFA%/ conversion | FFA%/conversion |
|---|---|---|
| time | rate(LM) | rate(LMB) |
| 8 h | 5%/95% | 4%/96% |
| 24 h | 3.4%/96.6% | 3.4%/96.6% |

It can be seen that the LM and LMB of the present invention lead to a further improved biodiesel conversion rate compared to TDLm2.

### Example 5: Expression of Mutants LMBW and LMBY in Pichia pastoris and Preparation of Enzyme Solution

The amino acid sequence of the mature peptide of LMB was introduced with the mutation F95W at amino acid position 95 to obtain the following amino acid sequence:

(LMBW, SEQ ID NO: 8)
DVSQDLFDQFNLFAQYSAAAYCAKNNRAPAGAIVTCRASICPEVEKADATFLYSFED
SGVGDVTGFLALDNTNRLIVLSFRGSRSIENWIGNLNWELKGIDDICSGCKGHAGFT
SSWRSVANTLTQQVQNAVREHPDYRVVFTGHSLGGALATVAGASLRGNKYDIDVFS
YGAPRVGNRAFAEFLTAQTGGTLYRITHTNDIVPRLPPREFGYSHSSPEYWITSGTLV
PVRRRDIVKVEGIDSTDGNNQPNTPSITAHFWYFGSIGTCL.

The DNA sequence was designed based on the codon preference of Pichia pastoris and the prepropeptide sequence of a-factor (derived from the DNA sequence of the commercial vector pPIC9K) was added. The following DNA encoding sequence was obtained:

(encoding sequence of LMBW, SEQ ID NO: 9)
ATGAGATTTCCTTCAATTTTTACTGCAGTTTTATTCGCAGCATCCTCCGCATTAGCT
GCTCCAGTCAACACTACAACAGAAGATGAAACGGCACAAATTCCGGCTGAAGCT
GTCATCGGTTACTCAGATTTAGAAGGGGATTTCGATGTTGCTGTTTTGCCATTTTC
CAACAGCACAAATAACGGGTTATTGTTTATAAATACTACTATTGCCAGCATTGCTG
CTAAAGAAGAAGGGGTATCTCTTGAGAAAAGAGAGGCTGAAGCTGATGTTTCTC
AAGATTTGTTTGATCAATTTAATTTGTTTGCTCAATATTCTGCTGCAGCTTATTGTG
CTAAAAATAATAGGGCTCCTGCTGGTGCTATTGTTACTTGTAGAGCTTCTATTTGT
CCTGAAGTTGAAAAAGCTGATGCTACTTTTTTGTATTCTTTTGAAGATTCTGGTGT
TGGTGATGTTACTGGTTTTTTGGCTTTGGATAATACTAATAGATTGATTGTTTTGTC
TTTTAGAGGATCTAGGTCAATTGAAAACTGGATTGGTAATTTGAATTGGGAATTGA
AAGGTATTGATGATATTTGTTCTGGTTGTAAAGGTCATGCTGGTTTTACTTCTTCTT
GGAGGTCTGTTGCTAATACTTTGACTCAACAAGTTCAAAATGCTGTTAGAGAACA
TCCTGATTATAGAGTTGTTTTTACTGGTCATTCTTTGGGTGGTGCTTTGGCTACTG
TTGCTGGTGCTTCTTTGAGAGGTAATAAATATGATATTGATGTTTTTTCTTATGGTG
CTCCAAGAGTTGGTAATAGAGCTTTTGCTGAATTTTTGACTGCTCAAACTGGTGG
TACTTTGTATAGAATTACTCATACTAATGATATTGTTCCAAGATTGCCACCAAGAGA
ATTTGGTTATTCTCATTCTTCTCCTGAATATTGGATTACTTCTGGTACTTTGGTTCC
TGTTAGAAGGAGAGATATTGTTAAAGTTGAAGGTATTGATTCTACTGATGGTAATAA
TCAACCAAATACTCCATCTATTACTGCTCATTTTTGGTATTTTGGTTCTATTGGTAC
TTGTTTGtaa

The DNA sequence was sent to Suzhou Genewiz Biotechnology Co., Ltd. for full gene synthesis and was cloned into pET-pAOm-plc vector through the Sacll and EcoRI sites to obtain the plasmid p-LMBW.

The amino acid sequence of the mature peptide of LMB was introduced with the mutation F95Y at amino acid position 95 to obtain the following amino acid sequence:

(LMBY, SEQ ID NO: 10)
DVSQDLFDQFNLFAQYSAAAYCAKNNRAPAGAIVTCRASICPEVEKADATFLYSFED
SGVGDVTGFLALDNTNRLIVLSFRGSRSIENWIGNLNYELKGIDDICSGCKGHAGFT
SSWRSVANTLTQQVQNAVREHPDYRVVFTGHSLGGALATVAGASLRGNKYDIDVFS
YGAPRVGNRAFAEFLTAQTGGTLYRITHTNDIVPRLPPREFGYSHSSPEYWITSGTLV
PVRRRDIVKVEGIDSTDGNNQPNTPSITAHFWYFGSIGTCL.

The DNA sequence was designed based on the codon preference of Pichia pastoris and the prepropeptide sequence of a-factor (derived from the DNA sequence of the commercial vector pPIC9K) was added. The following DNA sequence was obtained:

(encoding sequence of LMBY, SEQ ID NO: 11)
ATGAGATTTCCTTCAATTTTTACTGCAGTTTTATTCGCAGCATCCTCCGCATTAGCT
GCTCCAGTCAACACTACAACAGAAGATGAAACGGCACAAATTCCGGCTGAAGCT
GTCATCGGTTACTCAGATTTAGAAGGGGATTTCGATGTTGCTGTTTTGCCATTTTC
CAACAGCACAAATAACGGGTTATTGTTTATAAATACTACTATTGCCAGCATTGCTG
CTAAAGAAGAAGGGGTATCTCTTGAGAAAAGAGAGGCTGAAGCTGATGTTTCTC
AAGATTTGTTTGATCAATTTAATTTGTTTGCTCAATATTCTGCTGCAGCTTATTGTG
CTAAAAATAATAGGGCTCCTGCTGGTGCTATTGTTACTTGTAGAGCTTCTATTTGT
CCTGAAGTTGAAAAAGCTGATGCTACTTTTTTGTATTCTTTTGAAGATTCTGGTGT
TGGTGATGTTACTGGTTTTTTGGCTTTGGATAATACTAATAGATTGATTGTTTTGTC
TTTTAGAGGATCTAGGTCAATTGAAAACTGGATTGGTAATTTGAACTATGAATTGA
AAGGTATTGATGATATTTGTTCTGGTTGTAAAGGTCATGCTGGTTTTACTTCTTCTT
GGAGGTCTGTTGCTAATACTTTGACTCAACAAGTTCAAAATGCTGTTAGAGAACA
TCCTGATTATAGAGTTGTTTTTACTGGTCATTCTTTGGGTGGTGCTTTGGCTACTG
TTGCTGGTGCTTCTTTGAGAGGTAATAAATATGATATTGATGTTTTTTCTTATGGTG
CTCCAAGAGTTGGTAATAGAGCTTTTGCTGAATTTTTGACTGCTCAAACTGGTGG
TACTTTGTATAGAATTACTCATACTAATGATATTGTTCCAAGATTGCCACCAAGAGA
ATTTGGTTATTCTCATTCTTCTCCTGAATATTGGATTACTTCTGGTACTTTGGTTCC
TGTTAGAAGGAGAGATATTGTTAAAGTTGAAGGTATTGATTCTACTGATGGTAATAA
TCAACCAAATACTCCATCTATTACTGCTCATTTTTGGTATTTTGGTTCTATTGGTAC
TTGTTTGtaa.

The DNA sequence was sent to Suzhou Genewiz Biotechnology Co., Ltd. for full gene synthesis and was cloned into pET-pAOm-plc vector through the Sacll and EcoRI sites to obtain the plasmid p-LMBY.

p-LMBW and p-LMBY were linearized with Sall, and competent cells of Pichia pastoris GS115 were prepared by the LiAC method. The linearized p-LMBW and p-LMBY fragments were then transformed into GS115 competent cells by electroporation. The transformants were coated on a MGYS plate and cultured at 30° C. for 3 days. A large number of single clones on the plate were picked on a BMMY-olive oil screening plate, and positive clones with the best activity performance were picked out and named PLMBW and PLMBY.

PTDLm2, PLM, PLMB, PLMBW and PLMBY strains were first activated in liquid YPD, inoculated in BMGY, cultured overnight at 30° C., 220 rpm, and then transferred to BMGY medium (the initial OD600 was 6), and initially induced with 2% methanol. 1% methanol was added after 24 h and 32 h respectively, and 1% methanol was added after 48 h and 56 h respectively. After 72 h, the sample was concentrated with an ultrafiltration tube with a molecular weight cutoff of 10 KD to make the protein concentration reach 30 mg/ml.

The shake flask fermentation broth of PTDLm2, PLM, PLMB, PLMBWand PLMBY was taken and the protein concentration of the fermentation broth was determined using a modified Bradford protein concentration determination kit. The protein concentration of the fermentation broth of each strain is shown in FIG. 2.

The shake flask fermentation protein yields of LMBW and LMBY were 0.461 mg/ml and 0.498 mg/ml, respectively; compared with 0.25 mg/ml of TDLm2, they increased by 84% and 99% respectively; compared with 0.358 mg/ml of LMB, they increased by 29% and 39% respectively.

### Example 6: LMBW and LMBY Biodiesel Reaction Tests

The biodiesel preparation method is as described in Example 2.

The results of biodiesel production of LMBW and LMBY are shown in the following table.

| Reaction | FFA%/ conversion | FFA%/conversion |
|---|---|---|
| time | rate (LMBW) | rate (LMBY) |
| 8 h | 8%/92% | 4%/96% |
| 24 h | 3.4%/96.6% | 3.4%/96.6% |

After 24 hours of reaction, the FFA % of LMBW and LMBY can both be reduced to 3.4%, which meets the standard of biodiesel. Therefore, LMBW and LMBY have the advantage of cost saving compared with TDLM2, LM and LMB.

1. A lipase, comprising an amino acid sequence having a mutation relative to SEQ ID NO: 1 at one or more amino acid positions corresponding to positions 27, 38, 96, 111, 163, 254 and 256 of SEQ ID NO: 1; preferably, the mutation is one or more of D27R, G38A, D96E, D111A, G163K, D254S and A256T.

2. The lipase of claim 1, further comprising an additional mutation at one or more amino acid positions corresponding to positions 86, 93, 95 and 211 of SEQ ID NO: 1; preferably, the additional mutation is one or more of L86I, 193L and M95F; L86I, 193L, M95F and L211F; L86I, 193L, M95W and L211F; or L86I, 193L, M95Y and L211F.

3. The lipase of claim 1, comprising an amino acid sequence selected from the group consisting of SEQ ID NO: 2, 4, 6, 8 and 10, or a sequence having at least 90%, or at least 95% or at least 99% sequence identity thereto.

4. A nucleic acid molecule comprising:
(a) a nucleotide sequence encoding the lipase of claim 1, or a sequence having at least 90%, at least 95%, or at least 99% sequence identity thereto; and
(b) a nucleotide sequence complementary to the nucleotide sequence of (a),
preferably, the nucleic acid molecule comprises a nucleotide sequence selected from SEQ ID NO: 3, 5, 7, 9 and 11, or a sequence having at least 90%, or at least 95% or at least 99% sequence identity thereto.

5. A vector comprising the nucleic acid molecule of claim 4.

6. A host cell comprising the nucleic acid molecule of claim 4.

7. The host cell of claim 6, wherein the host cell is selected from the group consisting of a bacterial cell, a fungal cell, a mammalian cell, an insect cell and a plant cell, preferably, wherein the host cell is a fungal cell, more preferably a Pichia pastoris cell.

8. A method for producing a lipase, comprising expressing a nucleic acid molecule encoding the lipase of claim 1 in a host cell, and recovering the resulting polypeptide.

9. A composition comprising the lipase according to claim 1 or the fermentation broth, fermentation supernatant and/or fermentation concentrate of the host cell according to claim 6.

10. The fermentation broth, fermentation supernatant or fermentation concentrate of the host cell according to claim 6.

11. (canceled)

12. A method for preparing a lipase mutant, comprising introducing one or more mutations at amino acid positions of a starting lipase corresponding to positions 27, 38, 96, 111, 163, 254 and 256 of SEQ ID NO: 1, preferably, the mutation is one or more of D27R, G38A, D96E, D111A, G163K, D254S and A256T; optionally, the method further comprises introducing one or more mutations at amino acid positions of a starting lipase corresponding to positions 86, 93, 95 and 211 SEQ ID NO: 1, preferably, the mutation is one or more of L86I, 193L and M95F; L86I, 193L, M95F and L211F; L86I, 193L, M95Y and L211F; or L86I, 193L, M95W and L211F; preferably, the sequence of the starting lipase is as shown in SEQ ID NO: 1.

13. A method for preparing biodiesel, comprising incubating raw material oil, a low-carbon alcohol and a lipase according to claim 1 or a composition according to claim 9 or a fermentation liquid, a fermentation supernatant and/or a fermentation concentrate of a host cell according to claim 6 together to produce corresponding fatty acid ester, preferably, the raw material oil is selected from oil of oil crops, wild oil plants and aquatic plants such as engineered microalgae, animal fat and waste cooking oil, and the low-carbon alcohol is selected from methanol and ethanol.
